(19) United States
(12) Patent Application Publication
(10) Pub. No.: US 2024/0203579 A1
(43) Pub. Date: Jun. 20, 2024

(54) AT-HOME MEDICAL MONITORING SYSTEM FOR SENIOR CITIZENS

(71) Applicant: Marc Van Zyl, Peachtree City, GA (US)

(72) Inventor: Marc Van Zyl, Peachtree City, GA (US)

(21) Appl. No.: 18/082,624

(22) Filed: Dec. 16, 2022

(51) Int. Cl. G16H 40/67; G16H 70/60

(52) U.S. Cl. CPC G16H 40/67; G16H 70/60

(57) ABSTRACT

Methods, systems, and computer program products for operating an at-home medical monitoring system are provided. Aspects include operating the at-home medical monitoring system in a first operating mode and detecting an interaction with the at-home medical monitoring system by a patient. Aspects also include prompting the patient to obtain a biometric measurement corresponding to the one or more patient monitoring events based on a determination that one or more patient monitoring events are due. Aspects further include recording the biometric measurement and transmitting the measurement to a cloud storage device based on receiving the biometric measurement.

### BACKGROUND

The present invention generally relates to medical monitoring, and more specifically, to an at-home medical monitoring system for senior citizens.

Chronic diseases are conditions that last at least one year and require ongoing medical attention and limit daily activities and are a large and growing problem in the US. According to the Centers for Disease Control, chronic diseases cause 70% of all deaths, and chronic patients consume about 80% of healthcare resources. Most of the cost comes from hospital visits when the underlying condition triggers an attack or flare-up. For example, a patient with Chronic Obstructive Pulmonary Disease (COPD) costs about $700 to treat for a year—provided the patient experiences no flare-ups. However, in the event of a flare-up, a single hospitalization costs on average $7,000, and hospitalization costs for patients who stay 1 overnight average $11,700. Notably, most flare-ups can be identified using two pieces of biometric data, lung function tests and blood saturation levels, up to three days before patients are admitted to hospitals.

A key problem in managing chronic disease is effectively tracking s patient's condition and using this information to escalate care before a serious situation arises. In the above example, if a COPD patient's doctor could be alerted to an emerging flare-up days before it manifests, then the patient could be provided with stronger medication to prevent hospitalization. In the past few years, there has been a dramatic increase in the use of patient biometric sensor technology, for example, Bluetooth-enabled scales, thermometers, and pulse oximeters.

Unfortunately, the main group affected by chronic diseases, senior citizens, has been largely ignored by the increasing app and smartphone-based solutions that monitor critical biometrics to prevent chronic disease flare-ups or critical life-threatening episodes.

### SUMMARY

Embodiments of the present invention are directed to a computer-implemented method for operating an at-home medical monitoring system. A non-limiting example of the computer-implemented method operating the at-home medical monitoring system in a first operating mode and detecting an interaction with the at-home medical monitoring system by a patient. The method also includes prompting the patient to obtain a biometric measurement corresponding to the one or more patient monitoring events based on a determination that one or more patient monitoring events are due. The method further includes recording the biometric measurement and transmitting the measurement to a cloud storage device based on receiving the biometric measurement.

Embodiments of the present invention are directed to a computer-implemented method for configuring an at-home medical monitoring system. A non-limiting example of the computer-implemented method includes receiving, from a provider associated with a patient, a request for the at-home medical monitoring system and determining, based on the request, one or more biometric sensors for the at-home medical monitoring system and an operating mode of the at-home medical monitoring system. The method also includes pairing the one or more biometric sensors with the at-home medical monitoring system and configuring the at-home medical monitoring system based on the operating mode. The method also include shipping the at-home medical monitoring system and the one or more biometric sensor to the patient.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a block diagram of a computer system for use in implementing one or more embodiments of the present invention;

FIG. 2 depicts a block diagram of an at-home medical monitoring system for senior citizens one or more embodiments of the present invention;

FIG. 3 depicts a flow diagram of a method for configuring an at-home medical monitoring system for senior citizens according to one or more embodiments of the invention;

FIG. 4 depicts a flow diagram of a method for operating an at-home medical monitoring system for senior citizens according to one or more embodiments of the invention;

FIG. 5 depicts a flow diagram of another method for operating an at-home medical monitoring system for senior citizens according to one or more embodiments of the invention; and

FIG. 6 depicts an illustration of a user interface for ordering an at-home medical monitoring system for senior citizens according to one or more embodiments of the invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of #8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

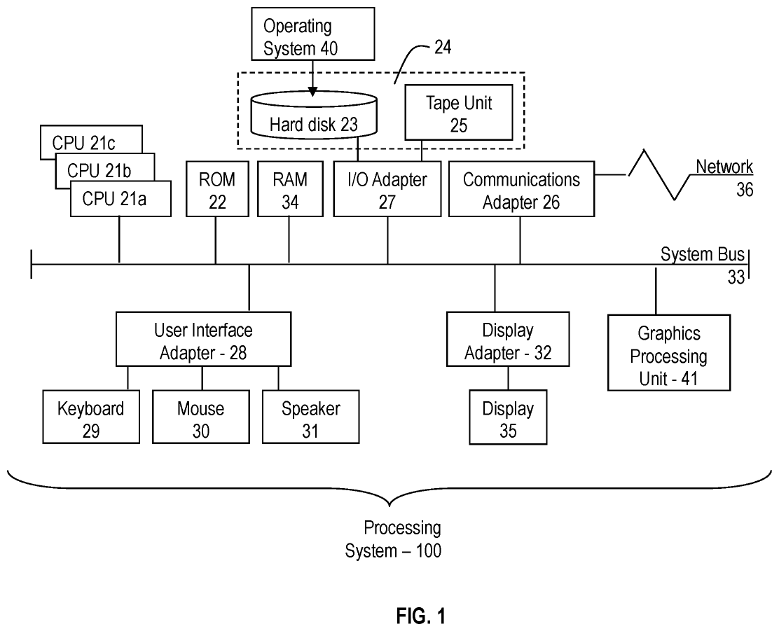

Referring to FIG. 1, there is shown an embodiment of a processing system 100 for implementing the teachings herein. In this embodiment, the system 100 has one or more central processing units (processors) 21a, 21b, 21c, etc. (collectively or generically referred to as processor(s) 21). In one or more embodiments, each processor 21 may include a reduced instruction set computer (RISC) microprocessor. Processors 21 are coupled to system memory 34 and various other components via a system bus 33. Read only memory (ROM) 22 is coupled to the system bus 33 and may include a basic input/output system (BIOS), which controls certain basic functions of system 300.

FIG. 1 further depicts an input/output (I/O) adapter 27 and a network adapter 26 coupled to the system bus 33. I/O adapter 27 may be a small computer system interface (SCSI) adapter that communicates with a hard disk 23 and/or tape storage drive 25 or any other similar component. I/O adapter 27, hard disk 23, and tape storage device 25 are collectively referred to herein as mass storage 24. Operating system 40 for execution on the processing system 300 may be stored in mass storage 24. A network adapter 26 interconnects bus 33 with an outside network 36 enabling data processing system 300 to communicate with other such systems. A screen (e.g., a display monitor) 35 is connected to system bus 33 by display adaptor 32, which may include a graphics adapter to improve the performance of graphics intensive applications and a video controller. In one embodiment, adapters 27, 26, and 32 may be connected to one or more I/O busses that are connected to system bus 33 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 33 via user interface adapter 28 and display adapter 32. A keyboard 29, mouse 30, and speaker 31 all interconnected to bus 33 via user interface adapter 28, which may include, for example, a Super I/O chip integrating multiple device adapters into a single integrated circuit.

In exemplary embodiments, the processing system 100 includes a graphics processing unit 41. Graphics processing unit 41 is a specialized electronic circuit designed to manipulate and alter memory to accelerate the creation of images in a frame buffer intended for output to a display. In general, the graphics processing unit 41 is very efficient at manipulating computer graphics and image processing and has a highly parallel structure that makes it more effective than general-purpose CPUs for algorithms where the processing of large blocks of data is done in parallel.

Thus, as configured in FIG. 1, the system 100 includes processing capability in the form of processors 21, storage capability including system memory 34 and mass storage 24, input means such as keyboard 29 and mouse 30, and output capability including speaker 31 and display 35. In one embodiment, a portion of system memory 34 and mass storage 24 collectively store an operating system to coordinate the functions of the various components shown in FIG. 1.

Turning now to an overview of the aspects of the invention, one or more embodiments of the invention address the above-described shortcomings of the prior art by providing an at-home medical monitoring system specifically designed for senior citizens that utilize behavior stacking to engage senior citizens and prompt them to perform the desired monitoring tasks. In exemplary embodiments, the at-home monitoring system is paired with multiple sensors that are configured to obtain the desired biometric data from a patient. The at-home monitoring system is configured to obtain the biometric data from the sensors and to save the data in a cloud-based server for analysis and intervention. The at-home monitoring system is also configured to leverage habit-building techniques called “habit stacking” to encourage monitoring compliance by linking a patient's habit of taking their biometrics reading with other daily routine habits.

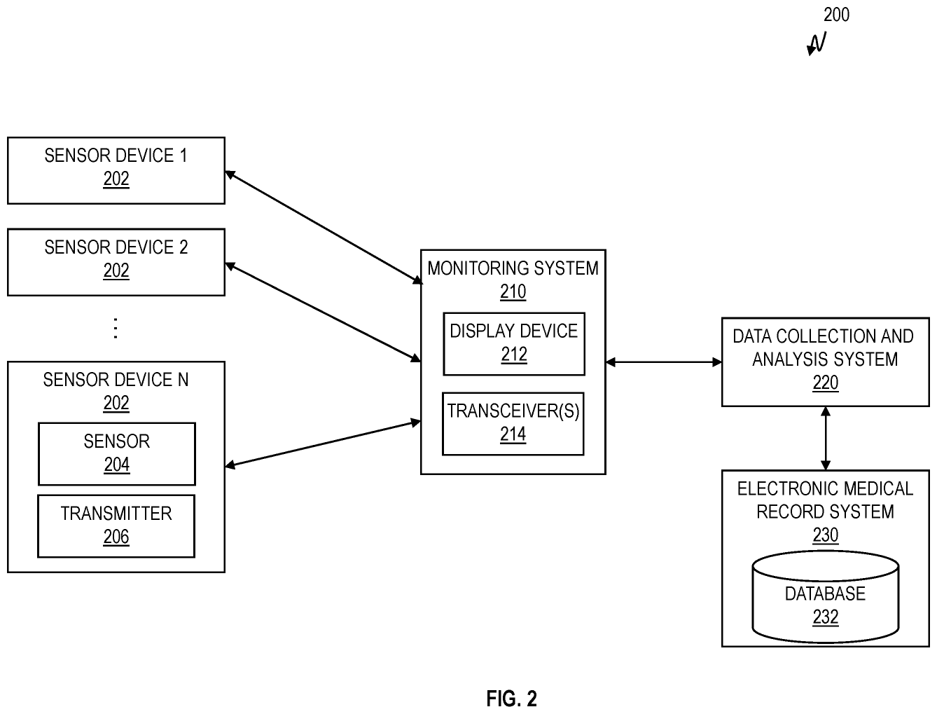

Turning now to a more detailed description of aspects of the present invention, FIG. 2 depicts a block diagram of a system 200 for at-home medical monitoring for senior citizens according to embodiments of the invention. As illustrated, the system 200 includes a monitoring system 210 that is in communication with a plurality of sensor devices 202. In one embodiment, the monitoring system 210 may be embodied in a processing system 100 as shown in FIG. 1. Each of the sensor devices 202 includes a sensor 204 and a transmitter 206. Each sensor 204 is configured to measure a particular type of biometric information of a user and the transmitter 206 is configured to provide the measured biometric data to the monitoring system 210. In exemplary embodiments, the types of biometric information that the sensor devices 202 can measure include, but are not limited to, heart rate, weight, heart rhythm, pulse oximetry, blood pressure, blood sugar, body mass index (BMI), electrical signals from the heart (EKG), and the like.

In exemplary embodiments, the monitoring system 210 includes, among other things, a display device 212 and a transceiver 214. The display device 212 is configured to facilitate communication with the patient and the transceiver 214 is configured to receive biometric information from the sensor devices 202 and to transmit the biometric data to a data collection and analysis system 220. In exemplary embodiments, the biometric data is transmitted to the data collection and analysis system 220 along with a patient identification number that is associated with the monitoring system 210.

In exemplary embodiments, the data collection and analysis system 220 is configured to receive the biometric data from the monitoring system 210 and to perform statistical analysis on the biometric data to identify potential anomalies or trends in the biometric data. In exemplary embodiments, the data collection and analysis system 220 is configured to store the biometric data in a database 232 of an electronic medical record (EMR) system 230 of a provider associated with the patient. In one embodiment, the data collection and analysis system 220 is further configured to create and transmit an alert to a provider, or emergency contact, based on a determination that the biometric data indicates a potentially dangerous anomaly. In this embodiment, the provider can proactively alert the patient about impending severe attacks and prevent the high costs, suffering, and even mortality involved in emergency hospitalizations.

In exemplary embodiments, the monitoring system 210 and the plurality of sensors 202 are sent to a patient in response to an order placed by a provider. The monitoring system 210 is paired with the plurality of sensors 202 prior to shipping the plurality of sensors 202 and the monitoring system 210 to the patient. The type of sensors 202 are determined based on the order placed by the provider. In addition, the monitoring system 210 is configured with an operating mode that is also determined by the order placed by the provider. In general, the operating mode of the monitoring system 210 is an operational function of the monitoring system 210 that is used to encourage engagement with the monitoring system 210 and to perform habit stacking techniques. The operating mode may include but is not limited to, a weather station mode, a crossword puzzle mode, a newspaper mode, and the like.

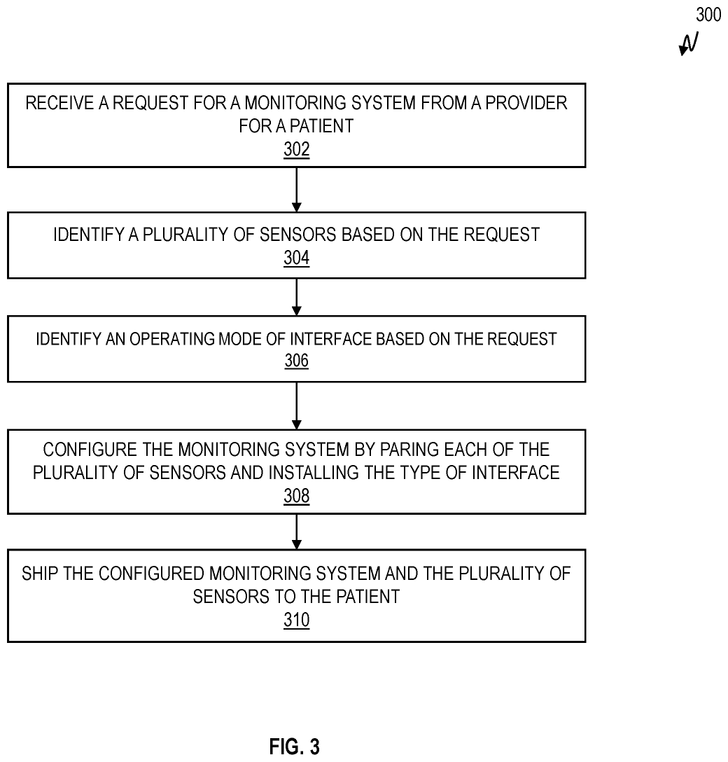

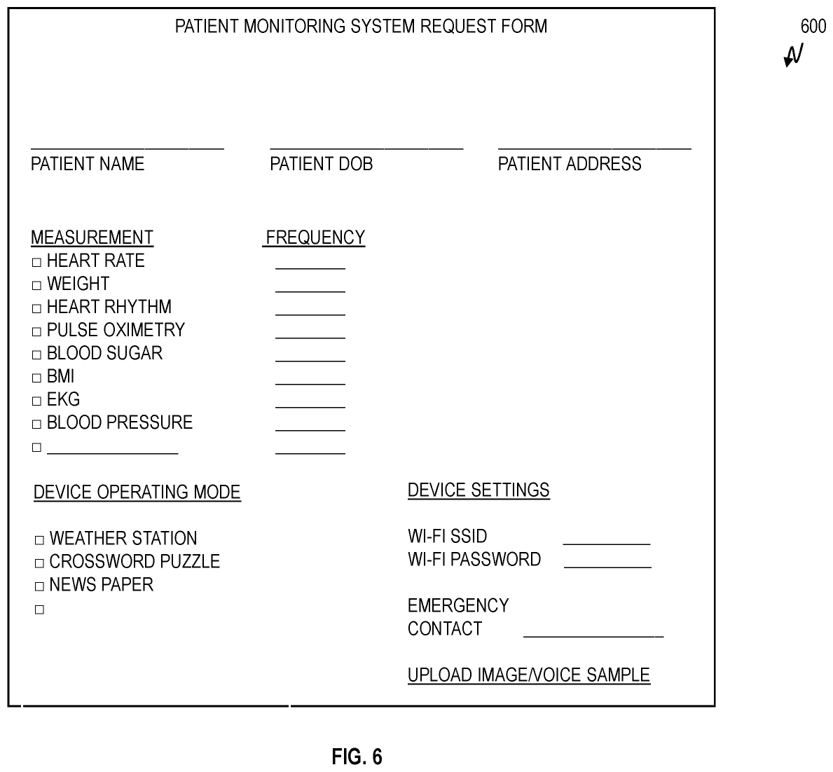

Referring now to FIG. 3, a flow diagram of a method 300 for configuring an at-home medical monitoring system for senior citizens according to one or more embodiments of the invention is shown. The method 300 includes receiving a request for a monitoring system from a provider for a patient, as shown at block 302. In one embodiment, the request may be submitted by a provider completing a request form, such as the one shown in FIG. 6. Next, as shown at block 304, the method 300 includes identifying a plurality of sensors based on the request. The method 300 also includes identifying an operating mode of interface based on the request, as shown at block 306.

Next, as shown at block 308, the method 300 includes configuring the monitoring system by paring each of the plurality of sensors and installing the type of interface. In exemplary embodiments, configuring the monitoring system can also include storing one or more how-to videos on the monitoring system that explain to a patient how to properly use each of the sensors that are paired with the monitoring system and how to troubleshoot the sensors. In other embodiments, configuring the monitoring system can also include preconfiguring one or more settings of the monitoring system for use in the patient's home. For example, the one or more settings can include an SSID and password of the patient's home Wi-Fi network. In another embodiment, configuring the monitoring system can also include providing a voice sample or facial image of the patient that can be used to ensure that the monitoring system can identify the patient in a multiuser environment. The method 300 concludes by shipping the configured monitoring system and the plurality of sensors to the patient, as shown at block 310.

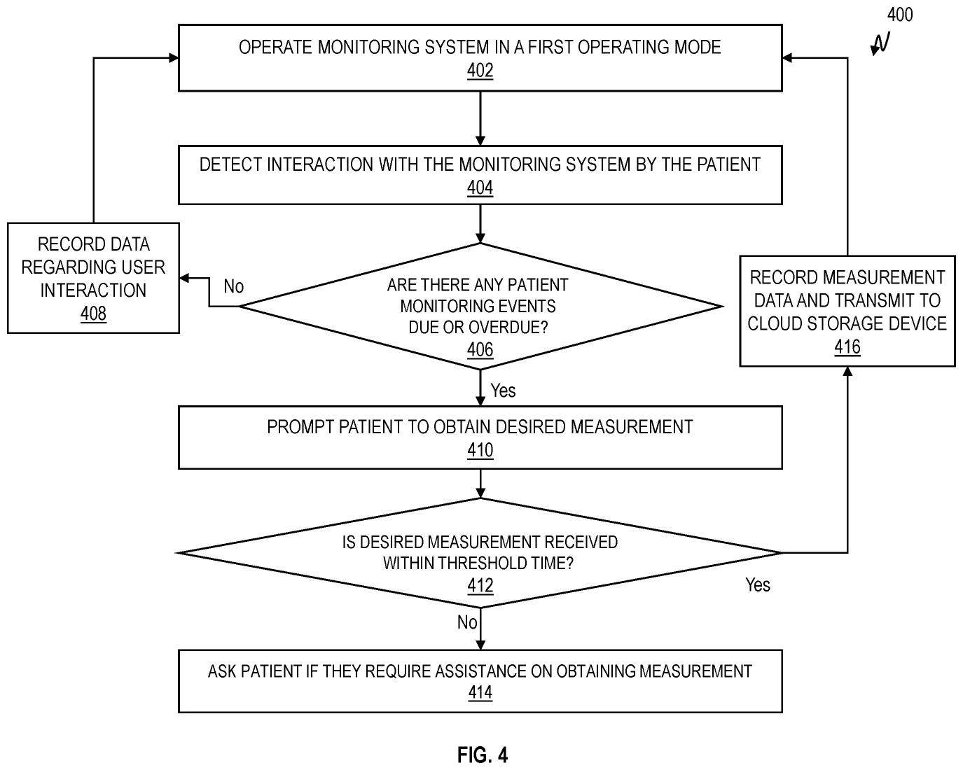

Referring now to FIG. 4, a flow diagram of a method 400 for operating an at-home medical monitoring system for senior citizens according to one or more embodiments of the invention is shown. As shown at block 402, the method 400 begins by operating the monitoring system in a first operating mode. For example, the first operating mode may be a weather station operating mode. Next, as shown at block 404, the method 400 includes detecting an interaction with the monitoring system by the patient. In one embodiment, detecting an interaction with the monitoring system by the patient includes detecting that a person is interacting with the monitoring system and determining whether the person is the patient based on one or more of voice recognition or facial recognition techniques.

Once the monitoring system determines that the patient is interacting with the monitoring system, the method 400 proceeds to decision block 406 and determines whether there are there any patient monitoring events due or overdue, i.e., does the patient need to perform any measurements with the paired sensors. In exemplary embodiments, the monitoring system is configured to collect each type of specified biometric data at a frequency set by a provider that ordered the monitoring system. For example, a patient may be required to obtain a pulse oximetry reading once per day and a body weight reading once per week.

Based on a determination that there are no patient monitoring events due or overdue, the method 400 proceeds to block 408 and records data regarding the user interaction with the monitoring system. In exemplary embodiments, the interaction data is used to identify usage patterns of the patient with the monitoring system to enhance the behavior stacking techniques utilized by the monitoring system. Based on a determination that there are patient monitoring events due or overdue, the method 400 proceeds to block 410 and prompts the patient to obtain desired measurement. Next, as shown at decision block 412, the method includes determining whether the desired measurement received within a threshold time since the prompt was provided. If the desired measurement was received within the threshold time, the method 400 proceeds to block 416 and records the measurement data and transmits the measurement data to a cloud storage device. If the desired measurement was not received within the threshold time, the method 400 proceeds to block 414 and asks the patient if they require assistance on obtaining measurement. If the patient indicates that they require assistance, a how-to video for using the proper sensor to obtain the desired measurement is shown by the monitoring system.

In exemplary embodiments, the monitoring system is configured to compare the biometric measurement to a threshold level corresponding to the patient. The threshold level may be set by a provider that ordered the monitoring system or may be based on a set deviation from a historical average of the patient's previously recorded biometric measurements. Based on determining that the biometric measurement exceeds the threshold level, the monitoring system is configured to transmit an alert to one of the provider and an emergency contact of the patient. The alert can include the name of the patient and the biometric measurement. In exemplary embodiments, the contact to whom the alert is sent is based on a severity level associated with the deviation of the biometric measurement.

In one embodiment, the monitoring system may further ask the patient if the sensor needs to be replaced, i.e., is the sensor battery dead, the sensor lost, etc. Based on a determination that the sensor needs to be replaced, the monitoring system is configured to re-order a sensor to replace the lost or damaged sensor. In exemplary embodiments, the replacement sensor is configured to be pre-paired with the monitoring system to facilitate easy use by the patient. In one embodiment, the replacement sensor is configured to have the same settings as the sensor that is being replaced to ensure that it will be automatically paired with the monitoring system.

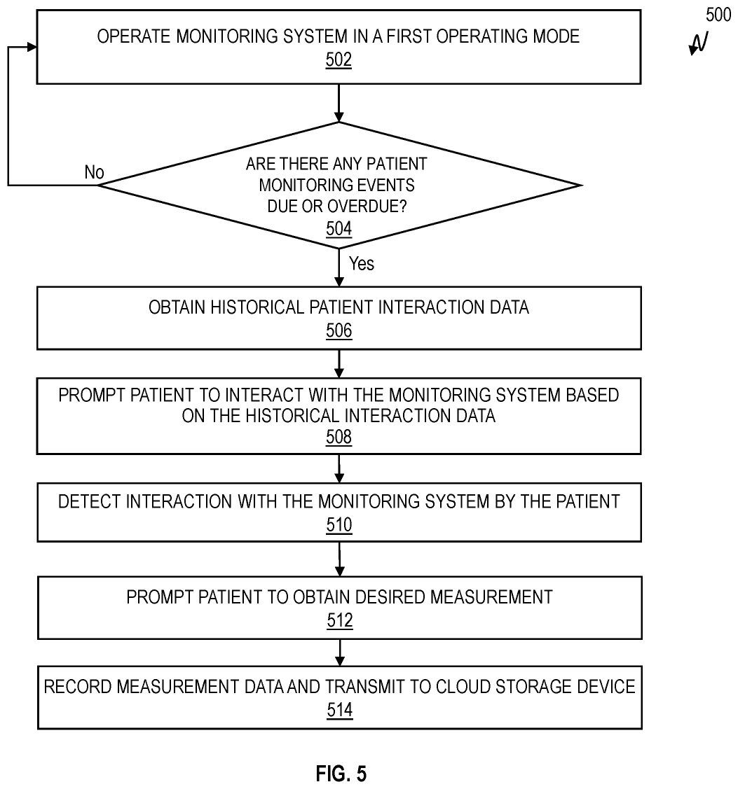

Referring now FIG. 5, a flow diagram of another method 500 for operating an at-home medical monitoring system for senior citizens according to one or more embodiments of the invention is shown. As shown at block 502, the method 500 begins by operating the monitoring system in a first operating mode. For example, the first operating mode may be a weather station operating mode. Next, as shown at decision block 504, the method 500 includes determining whether there are there any patient monitoring events due or overdue, i.e., does the patient need to perform any measurements with the paired sensors. If there are no patient monitoring events due or overdue, the method 500 returns to block 502. Otherwise, the method 500 proceeds to block 506 and obtains historical patient interaction data for the monitoring system.

Next, as shown at block 508, the method 500 includes prompting the patient to interact with the monitoring system based on the historical interaction data. In one embodiment, the prompt includes playing a video and/or a sound to get the attention of a user at a time during which the historical data indicates that the user is likely to interact with the monitoring system. In one embodiment, a camera or microphone of the monitoring system are configured to detect a presence of an individual nearby and the monitoring system provides the prompt based on a determination that an individual is in proximity to the monitoring system. In exemplary embodiments, the monitoring systems plays, or displays, the prompts at a time determined based on the historical usage pattern of the monitoring system by the patient. For example, if the patient typically interacts with the monitoring system between eight and nine in the morning, the monitoring system may provide a prompt to the patient during this time period.

As shown at block 510, the method 500 includes detecting an interaction with the monitoring system by the patient. In one embodiment, detecting an interaction with the monitoring system by the patient includes detecting that a person is interacting with the monitoring system and determining whether the person is the patient based on one or more of a voice recognition or facial recognition techniques. Once it is determined that the patient is interacting with the monitoring system, the method 500 proceeds to block 512 and prompts the patient to obtain desired measurement. In one embodiment, prompting the patient to obtain the biometric measurement includes displaying an image of a biometric sensor that the patient should use to obtain the biometric measurement. The method 500 concludes at block 416 by recording the measurement data and transmitting the measurement data to a cloud storage device.

Referring now to FIG. 6, an illustration of a user interface (UI) 600 for ordering at-home medical monitoring system for senior citizens according to one or more embodiments of the invention is shown. As illustrated, the UI 600 is configured to receive data from a provider that is used to configure an at-home medical monitoring system for a patient. The UI 600 receives a patient name, date of birth, and mailing address that is used to ship the monitoring system. The UI 600 receives an indication of a type of measurements that the provider would like the monitoring system to obtain and the frequency that each measurement should be taken. In exemplary embodiments, the types of measurements that the provider selects are used to determine what types of sensors are to be paired with, and shipped with, the monitoring system. In addition, the UI 600 is configured to receive a selection of a device operating mode that is used to configure the monitoring system.

In exemplary embodiments, the UI 600 is also configured to receive an image of the patient and/or a voice sample of the patient, which can be used by the monitoring system to identify the patient in a multiuser environment. In one embodiment, the UI 600 is configured to receive an emergency contact that the monitoring system can contact in the event that a measured piece of biometric information indicates a medical emergency. In another embodiment, the UI 600 is configured to receive WiFi setting of a WiFi network in a patient's home that is used to pre-configure the monitoring system for use in the patient's home prior to shipping the monitoring system.

Additional processes may also be included. It should be understood that the processes depicted in FIGS. 3, 4 and 5 represent illustrations, and that other processes may be added or existing processes may be removed, modified, or rearranged without departing from the scope and spirit of the present disclosure.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method for operating an at-home medical monitoring system, the method comprising:
operating the at-home medical monitoring system in a first operating mode;
detecting an interaction with the at-home medical monitoring system by a patient;
based on a determination that one or more patient monitoring events are due, prompting the patient to obtain a biometric measurement corresponding to the one or more patient monitoring events; and
based on receiving the biometric measurement, recording the biometric measurement and transmitting the biometric measurement to a cloud storage device.

2. The computer-implemented method of claim 1, further comprising recording data regarding the interaction by the at-home medical monitoring system.

3. The computer-implemented method of claim 2, further comprising identifying a usage pattern of the patient for the at-home medical monitoring system based on the recorded data.

4. The computer-implemented method of claim 3, further comprising:
based on a determination that one or more patient monitoring events are due and that the patient is not interacting with the at-home medical monitoring system, prompting the patient to interact with the at-home medical monitoring system at a time determined based on the usage pattern.

5. The computer-implemented method of claim 1, wherein detecting the interaction with the at-home medical monitoring system by the patient comprises:
determining that a person is interacting with the at-home medical monitoring system by a patient; and
determining that the person is the patient based on one or more of a facial recognition and a voice recognition.

6. The computer-implemented method of claim 1, further comprising:
based on a determination that the biometric measurement corresponding to the one or more patient monitoring events has not been received during a time period after the prompting, asking the patient whether assistance is required in obtaining the biometric measurement;
in response to the patient requesting assistance in obtaining the biometric measurement, displaying an instructional video on how to obtain the biometric measurement.

7. The computer-implemented method of claim 1, wherein prompting the patient to obtain the biometric measurement includes displaying an image of a biometric sensor that the patient should use to obtain the biometric measurement.

8. The computer-implemented method of claim 7, further comprising:
based on a determination that the biometric measurement corresponding to the one or more patient monitoring events has not been received during a time period after the prompting, asking the patient whether the biometric sensor needs to be replaced; and
in response to the patient requesting that the biometric sensor be replaced, transmitting a request for a replacement biometric sensor, wherein the replacement biometric sensor will be automatically paired with the at-home medical monitoring system.

9. The computer-implemented method of claim 1, further comprising:
comparing the biometric measurement to a threshold level corresponding to the patient; and
based on determining that the biometric measurement exceeds the threshold level transmitting an alert to one of a provider and an emergency contact of the patient, wherein the alert includes a name of the patient and the biometric measurement.

10. The computer-implemented method of claim 1, wherein the first operating mode is one of a weather station mode, a newspaper mode, and a crossword puzzle mode and wherein the operating mode is determined based on a request for the at-home medical monitoring system from a provider of the patient.

11. The computer-implemented method of claim 1, wherein each of the one or more patient monitoring events are determined based on a request for the at-home medical monitoring system from a provider of the patient and wherein the request includes a plurality of desired biometric measurements and a frequency for each of the plurality of desired biometric measurements.

12. The computer-implemented method of claim 1, wherein each of the one or more patient monitoring include an identification of a biometric sensor for obtaining the biometric measurement.

13. A computer program product comprising a computer readable storage medium having program instructions embodied therewith, the program instructions executable by a processor to cause the processor to perform operations comprising:
operating a at-home medical monitoring system in a first operating mode;
detecting an interaction with the at-home medical monitoring system by a patient;
based on a determination that one or more patient monitoring events are due, prompting the patient to obtain a biometric measurement corresponding to the one or more patient monitoring events; and
based on receiving the biometric measurement, recording the biometric measurement and transmitting the biometric measurement to a cloud storage device.

14. The computer program product of claim 13, wherein the operations further comprise recording data regarding the interaction by the at-home medical monitoring system.

15. The computer program product of claim 14, wherein the operations further comprise identifying a usage pattern of the patient for the at-home medical monitoring system based on the recorded data.

16. The computer program product of claim 15, wherein the operations further comprise:
based on a determination that one or more patient monitoring events are due and that the patient is not interacting with the at-home medical monitoring system, prompting the patient to interact with the at-home medical monitoring system at a time determined based on the usage pattern.

17. The computer program product of claim 13, wherein detecting the interaction with the at-home medical monitoring system by the patient comprises:
determining that a person is interacting with the at-home medical monitoring system by a patient; and
determining that the person is the patient based on one or more of a facial recognition and a voice recognition.

18. The computer program product of claim 13, wherein the operations further comprise:
based on a determination that the biometric measurement corresponding to the one or more patient monitoring events has not been received during a time period after the prompting, asking the patient whether assistance is required in obtaining the biometric measurement;
in response to the patient requesting assistance in obtaining the biometric measurement, displaying an instructional video on how to obtain the biometric measurement.

19. The computer program product of claim 13, wherein prompting the patient to obtain the biometric measurement includes displaying an image of a biometric sensor that the patient should use to obtain the biometric measurement.

20. A computer-implemented method for configuring an at-home medical monitoring system, the method comprising:
receiving, from a provider associated with a patient, a request for the at-home medical monitoring system;
determining, based on the request, one or more biometric sensors for the at-home medical monitoring system and an operating mode of the at-home medical monitoring system;
pairing the one or more biometric sensors with the at-home medical monitoring system;
configuring the at-home medical monitoring system based on the operating mode; and
shipping the at-home medical monitoring system and the one or more biometric sensor to the patient.
